(19) United States
(12) Patent Application Publication
(10) Pub. No.: US 2018/0273543 A1
(43) Pub. Date: Sep. 27, 2018

(54) METHOD FOR PRODUCING LAMELLARIN AND DERIVATIVE THEREOF

(71) Applicant: Tunghai University, Taichung City (TW)

(72) Inventors: Ding-Yah YANG, Taichung City (TW); Kiran B. MANJAPPA, Taichung City (TW)

(21) Appl. No.: 15/865,859

(22) Filed: Jan. 9, 2018

(30) Foreign Application Priority Data
Mar. 27, 2017 (TW) 106110084

(51) Int. Cl. C07D 491/052

(52) U.S. Cl. CPC C07D 491/052

(57) ABSTRACT

The present application discloses a method for producing lamellarin and a derivative thereof, which is able to greatly shorten the synthesis path of the lamellarin and the derivative thereof, and to improve the yield of the lamellarin and the derivative thereof, so as to increase use of the lamellarin or the derivative thereof in pharmaceutical industry.

### TECHNICAL FIELD

The present application relates to a method for the synthesis of an organic compound, particularly to a method for producing a lamellarin and a derivative thereof.

### BACKGROUND

Lamellarin D is a natural product isolated from marine invertebrates and has a unique pyrrolocoumarin structure. It is known from study that the lamellarin D has a variety of biological activities, for example, cytotoxicity to a variety of drug-resistant tumor cell lines (Kluza, J. et al., 2006; Ballot, C. et al., 2010) and as a DNA topoisomerase I inhibitor (Facompre', M. et al., 2003; Macro, et al., 2005; Khiati, S. et al., 2014). Although lamellarin D has many biological activities, lamellarin D is difficult to be obtained in large quantities in nature. Therefore, for nearly 30 years, organic medicinal chemists have devoted much effort to study the synthesis of lamellarin D and its related alkaloids.

Furthermore, synthesis strategies of lamellarin D can be generally divided into two categories, one of which is to construct a pyrrole core as a critical step; the other is to incorporate functionalization of the pre-existing pyrrole as aims. In the past, however, the pyrrole ring was cyclized directly to a functionalized coumarin derivative to provide a pentacyclic core of the lamellarin, however, the yield of this method was only 5 to 6% (Ploypradith, P. et. al, 2004).

Thus, a production method of the lamellarin and its derivative that is fast and in great quantity is desired in the prior art.

### SUMMARY

It is one objective of the present application to provide a method for producing a lamellarin and a derivative thereof that is able to greatly shorten the synthesis path of the lamellarin and the derivative thereof.

It is another objective of the present application to provide a method for producing a lamellarin and a derivative thereof that is able to improve the yield of the lamellarin and the derivative thereof so as to increase the use of the lamellarin and the derivative thereof in the pharmaceutical industry.

In order to achieve the above objectives, the present application provides a compound presented by formula (I):

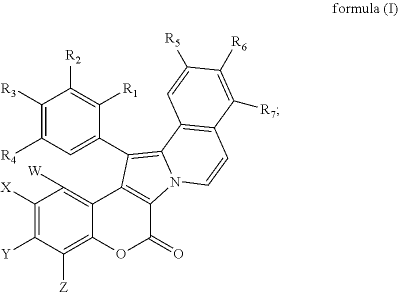

the method comprises: performing intermolecular cyclization reaction between a 3-nitrocoumarin derivative and a papaverine derivative under a pre-determined reaction condition to acquire a lamellarin or a derivative thereof, in which:

the reaction condition comprises use of at least one solvent and heating, and the solvent is an aromatic hydrocarbon, an ether, or a halogenated hydrocarbon; for example, the solvent is xylene, toluene, tetrahydrofuran, or 1,2-dichloroethane;

W is hydrogen, chlorine, bromine, fluorine, methoxy, methyl, or cyano;

X is hydrogen, benzyloxy, hydroxyl, chlorine, bromine, fluorine, trifluoromethyl, methoxy, methyl, or cyano;

Y is hydrogen, benzyloxy, hydroxyl, chlorine, bromine, fluorine, trifluoromethyl, methoxy, methyl, or cyano;

Z is hydrogen, benzyloxy, hydroxyl, chlorine, bromine, fluorine, trifluoromethyl, methoxy, methyl, or cyano;

R1, R2, and R7 are hydrogen respectively;

R3, R5, and R6 are methoxy, benzyloxy, hydroxyl, or hydrogen respectively, and R3, R5, and R6 are different functional groups; and

R4 is methoxy, hydroxyl, or hydrogen.

Further, a structure of the 3-nitrocoumarin derivative is represented by formula (II):

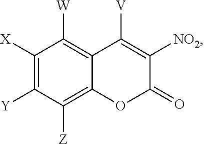

in which, V is hydrogen or chlorine.

For example, 3-nitrocoumarin derivative is 3-nitrocoumarin, 6-methoxy-7-benzyloxy-3-nitrocoumarin, or 6,7-dimethoxy-3-nitrocoumarin.

A structure of the papaverine derivative is represented by formula (III):

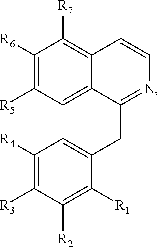

in which:

for example, the papaverine derivative is 1-(3,4-dimethoxybenzyl)-6,7-dimethoxyisoquinoline or 1-(3-benzyloxy-4-methoxy)-6-methoxy-7-benzyloxyisoquinoline.

Preferably, the production method of the compound represented by formula (I) disclosed by the present application is performed in a sealed space, and the sealed space can be formed by a sealed tube.

In the production method of the compound represented by formula (I) disclosed by the present application, a reaction temperature is at least 120° C., for example, 120° C., 130° C., 140° C., 150° C., 160° C., 170° C., and preferably at between 120° C. and 160° C.

In the production method of the compound represented by formula (I) disclosed by the present application, the reaction condition further comprises addition of an alkaline substance. For example, the alkaline substance is sodium bicarbonate, cesium carbonate, sodium carbonate, or potassium carbonate.

In the production method of the compound represented by formula (I) disclosed by the present application, when Z in the compound represented by formula (II) is chlorine, the reaction condition further comprises heating and addition of a catalyst accepting foreign electron pairs, in which, the catalyst accepting foreign electron pairs is aluminum chloride.

The production method of the compound represented by formula (I) disclosed by the present application in order to acquire lamellarin D and a derivative thereof, comprises the following steps:

step a: performing intermolecular cyclization reaction between 6-methoxy-7-benzyloxy-3-nitrocoumarin and 1-(3-benzyloxy-4-methoxy)-6-methoxy-7-benzyloxyisoquinoline in a sealed alkaline environment to acquire a compound having a pentacyclic core;

step b: performing catalytic hydrogenolysis on the compound having the pentacyclic core under a hydrogen gas atmosphere to acquire the compound represented by formula (I); in which:

a catalyst is palladium hydroxide on carbon or palladium on carbon;

X, R2, and R3 represent methoxy respectively;

Y, R1, and R4 represent hydroxyl respectively; and

R5 represents hydrogen.

Preferably, the reaction temperature of step a is 130° C. above.

The production method of the compound represented by formula (I) disclosed by the present application in order to acquire lamellarin D and a derivative thereof, comprises: performing intermolecular cyclization reaction between 6,7-dimethoxy-3-nitrocoumarin and 1-(3,4-dimethoxybenzyl)-6,7-dimethoxyisoquinoline in a sealed environment to acquire the compound represented by formula (I), in which:

X, Y, R1, R2, R3, and R4 represent methoxy respectively; and

R5 represents hydrogen.

Preferably, the reaction temperature is 150° C. above, for example 160° C.

The production method of the compound represented by formula (I) disclosed by the present application in order to acquire lamellarin H and a derivative thereof, comprises the following steps:

step a: performing intermolecular cyclization reaction between 6,7-dimethoxy-3-nitrocoumarin and 1-(3,4-dimethoxybenzyl)-6,7-dimethoxyisoquinoline in a sealed environment to acquire a compound having a pentacyclic core; and

step b: demethylating the compound having the pentacyclic core acquired from step a to remove methoxy therefrom so as to acquire the compound represented by formula (I); in which:

X, Y, R1, R2, R3, and R4 represent hydroxyl respectively; and

R5 represents hydrogen.

Further, in step b, a boron tribromide/dichloromethane solution is added for demethylation.

The production method of the compound represented by formula (I) disclosed by the present application in order to acquire a lamellarin and a derivative thereof, comprises: performing intermolecular cyclization reaction between the compound represented by formula (II) and the compound represented by formula (III) in a sealed environment to acquire the compound represented by formula (I), in which:

W is hydrogen;

X is methoxy, hydrogen, or hydroxyl;

Y is methoxy, hydroxyl, hydrogen, or chlorine;

Z is methoxy, hydroxyl, or hydrogen;

R1, R2, and R7 are hydrogen respectively; and

R3, R4, R5, and R6 are methoxy, hydroxyl, and hydrogen, and R3, R4, R5, and R6 are different functional groups.

Further, in the above production method, the demethylation is performed after the intermolecular cyclization reaction is completed to acquire the compound represented by formula (I), in which:

W is hydrogen;

X is hydrogen or hydroxyl;

Y is hydroxyl, hydrogen, or chlorine;

Z is hydroxyl or hydrogen;

R1, R2, and R7 are hydrogen respectively; and

R3, R4, R5, and R6 are hydroxyl or hydrogen, and R3, R4, R5, and R6 are different functional groups.

Further, the reaction condition further comprises addition of an alkaline substance, in which, the alkaline substance is sodium bicarbonate, cesium carbonate, sodium carbonate, and potassium carbonate, and the reaction temperature is between 150 and 160° C.

### DETAILED DESCRIPTION OF THE EMBODIMENTS

Unless otherwise defined, scientific terminology used in the present application should be construed according to the understanding of the ordinary technical staff in the technical field to which the present application pertains.

The coupling reaction disclosed in the present application is an organic chemical reaction. Herein, the coupling reaction allows intramolecular cyclization of two compounds or chemical units under the help of heating or/and catalyst to acquire a lamellarin or a derivative thereof.

Suzuki coupling reaction disclosed in the present application refers to a coupling reaction of an organoboron compound and an organic halide catalyzed by palladium.

Lamellarins or derivatives thereof disclosed by the present application have a common structure represented by formula (I):

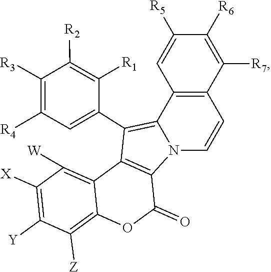

in which:

W is hydrogen, chlorine, bromine, fluorine, methoxy, methyl, or cyano;

X is hydrogen, benzyloxy, hydroxyl, chlorine, bromine, fluorine, trifluoromethyl, methoxy, methyl, or cyano;

Y is hydrogen, benzyloxy, hydroxyl, chlorine, bromine, fluorine, trifluoromethyl, methoxy, methyl, or cyano;

Z is hydrogen, benzyloxy, hydroxyl, chlorine, bromine, fluorine, trifluoromethyl, methoxy, methyl, or cyano;

R1, R2, and R7 are hydrogen respectively;

R3 is methoxy, benzyloxy, hydroxyl, or hydrogen;

R5 is methoxy, benzyloxy, hydroxyl, or hydrogen;

R6 is methoxy, benzyloxy, hydroxyl, or hydrogen; and

R4 is methoxy, hydroxyl, or hydrogen.

Lamellarin family includes: lamellarin D, lamellarin H, etc., for example, lamellarin D has a structural formula of

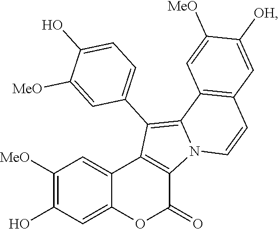 [SMILES: COc1cc(-c2c3c4cc(OC)c(O)cc4oc(=O)c3n3ccc4cc(O)c(OC)cc4c23)ccc1O]

lamellarin H has a structural formula of

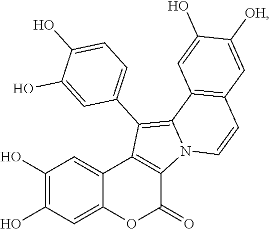 [SMILES: O=c1oc2cc(O)c(O)cc2c2c(-c3ccc(O)c(O)c3)c3c4cc(O)c(O)cc4ccn3c12]

lamellarin 501 has a structural formula of

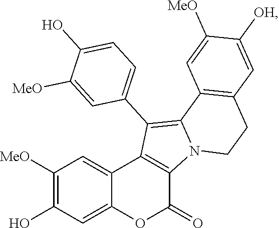 [SMILES: COc1cc(-c2c3n(c4c(=O)oc5cc(O)c(OC)cc5c24)CCc2cc(O)c(OC)cc2-3)ccc1O]

and lamellarin D trimethyl ether has a structural formula of

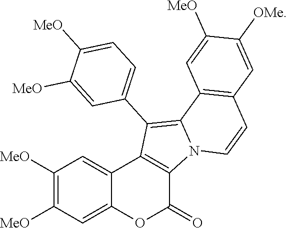 [SMILES: COc1ccc(-c2c3c4cc(OC)c(OC)cc4oc(=O)c3n3ccc4cc(OC)c(OC)cc4c23)cc1OC]

Structural formulas of the lamellarin derivatives include:

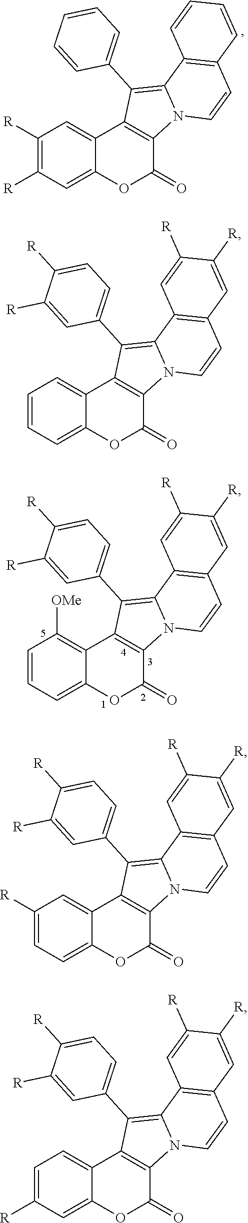

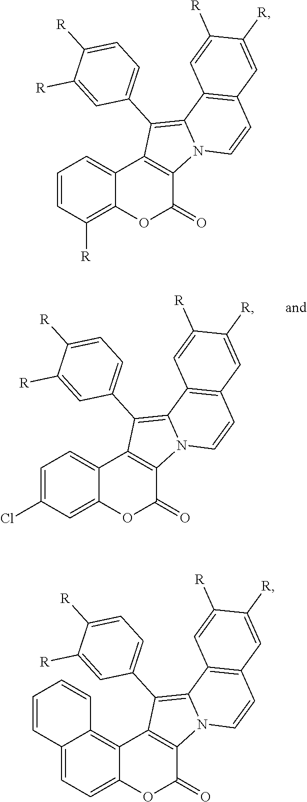

in which, R is OMe or OH.

The 3-nitrocoumarin derivative disclosed by the present application has a formula (II) of

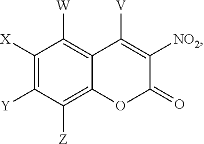

in which:

V is hydrogen or chlorine;

W is hydrogen, chlorine, bromine, fluorine, methoxy, methyl, or cyano;

X is hydrogen, benzyloxy, hydroxyl, chlorine, bromine, fluorine, trifluoromethyl, methoxy, methyl, or cyano;

Y is hydrogen, benzyloxy, hydroxyl, chlorine, bromine, fluorine, trifluoromethyl, methoxy, methyl, or cyano;

Z is hydrogen, benzyloxy, hydroxyl, chlorine, bromine, fluorine, trifluoromethyl, methoxy, methyl, or cyano.

For example, the structural formula of the 3-nitrocoumarin derivative is

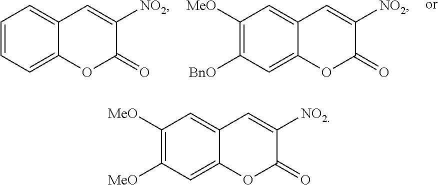

Further, the 3-nitrocoumarin derivative is a pure compound prepared by chemical synthesis and purification, for example, a toluene solution containing substituted o-hydroxybenzaldehyde (1 equivalent), ethyl nitroethylacetate (1.2 equivalents), and piperidine (1.2 equivalents) is heated at a temperature of approximately 110° C. for 6 hrs, after being cooled, a solid substance is filtered therefrom, thereafter, the solid substance is washed by ethyl acetate/hexane, dried to remove ethyl nitroethylacetate, and recrystallized to form the pure 3-nitrocoumarin.

The papaverine derivative disclosed by the present application has a formula (III) of

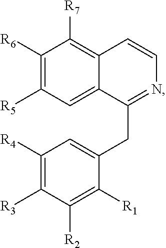

in which:

R1, R2, and R7 are hydrogen respectively;

R3 is methoxy, benzyloxy, hydroxyl, or hydrogen;

R5 is methoxy, benzyloxy, hydroxyl, or hydrogen;

R6 is methoxy, benzyloxy, hydroxyl, or hydrogen;

R4 is methoxy, hydroxyl, or hydrogen.

For example, the structural formula of the papaverine derivative is

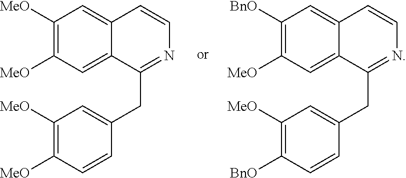 [SMILES: COc1ccc(Cc2nccc3cc(OC)c(OC)cc23)cc1OC ; "or" ; COc1cc(Cc2nccc3cc(OCc4ccccc4)c(OC)cc23)ccc1OCc1ccccc1]

The present application provides a production method of a lamellarin or a derivative thereof, the main step of which includes performing intermolecular cyclization reaction between a 3-nitrocoumarin derivative and a papaverine derivative, so as to effectively shorten the synthesis steps and improve the yield. The present application will be described in detail by illustrating a plurality of embodiments accompanying with schemes.

A first embodiment of the present application provides a method for preparing a core structure of a lamellarin, which comprises reactions represented by reaction scheme (I):

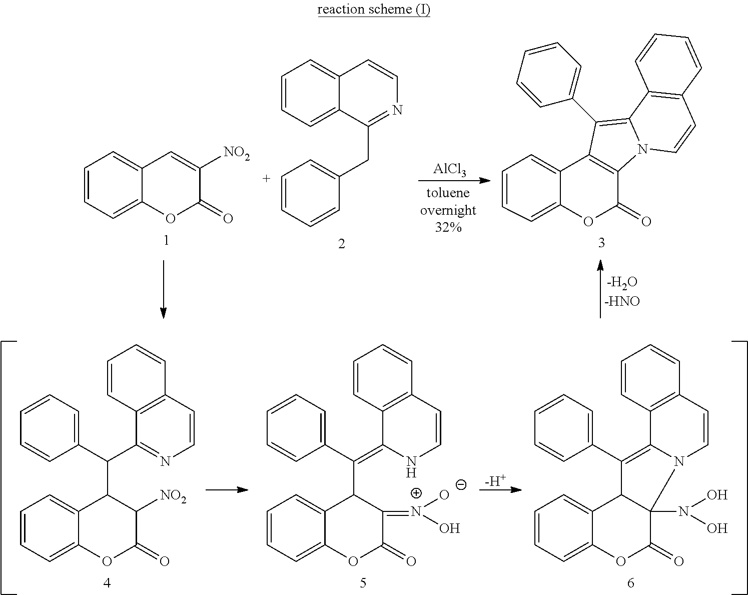

Particularly, in the reaction scheme (I), the 3-nitrocoumarin represented by formula (1) and 1-benzylisoquinoline represented by formula (2) are directly coupled, that is, a mixture of 3-nitrocoumarin and 1-benzylisoquinoline is refluxed by 1 equivalent of an aluminum chloride/toluene solution for reaction overnight to acquire a compound represented by formula (3), which is the core structure of lamellarin and has a yield of 32 wt. %.

In which, because of the reaction scheme (I) involves the addition of the aluminum chloride, a Michael Adduct represented by formula (4) is yielded in the reaction process. The compound presented by formula (4) is further isomerized to yield an enamine represented by formula (5), and the compound represented by formula (5) is intramolecularly cyclized via nucleophilic addition of amine nitrogen so as to yield a cyclized dihydroxylamine represented by formula (6). Water and nitroxylic acid are removed from the compound represented by the formula (6) to yield the compound represented by formula (3).

In embodiments of the present application, a method for preparing a lamellarin and a derivative thereof is further provided, a main step of which includes allowing 3-nitrocoumarin and a papaverine derivative for intermolecular cyclization reaction.

A second embodiment of the present application provides a method for preparing a core structure of lamellarin, which comprises reactions represented by reaction scheme (I):

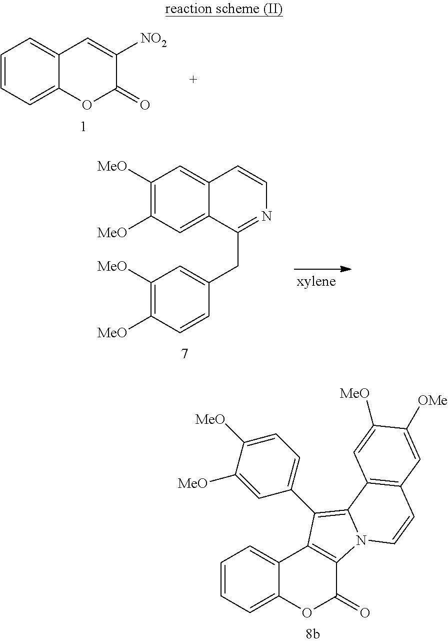 [SMILES: O=c1oc2ccccc2cc1[N+](=O)[O-] ; COc1ccc(Cc2nccc3cc(OC)c(OC)cc23)cc1OC ; COc1ccc(-c2c3c4ccccc4oc(=O)c3n3ccc4cc(OC)c(OC)cc4c23)cc1OC]

It is known from the reaction scheme (II) that in the second embodiment of the present application, a lamellarin derivative represented by formula (8b) is synthesized by 3-nitrocoumarin represented by formula (I) and papaverine (1-(3,4-dimethoxybenzyl)-6,7-dimethoxyisoquinoline) represented by formula (7) in an environment containing xylene, in which, the synthesis yield performed in a sealed tube or a close system will be higher than that in an open environment.

Further, Table 1 hereinbelow lists comparisons of yields of the lamellarin derivative under different reaction condition, which indicates that the most suitable reaction condition for production of the lamellarin or the derivative thereof includes: a sealed circulating environment, an organic solvent, alkaline environment, and heating, in which:

the organic solvent is an aromatic hydrocarbon, an ether, and a halogenated hydrocarbon, such as, xylene, toluene, tetrahydrofuran, and 1,2-dichloroethane.

The alkaline environment refers to addition of an alkali substance, such as, sodium bicarbonate, cesium carbonate, sodium carbonate, potassium carbonate, etc., and in condition that sodium bicarbonate is added, a relatively good yield can be acquired.

The reaction temperature should be heated to 120° C. above, for example, between 120 and 130° C., or between 150 and 160° C.

TABLE 1
Relationship between the reaction condition
and the yield of the lamellarin derivative

| | | | Time | Temperature | Separation |
|---|---|---|---|---|---|
| Number | Alkali | Equivalent | (hr) | (° C.) | yield (%) |
| 1 | NaHCO₃ | 1.2 | 16 | 120 | 18 |
| 2 | NaHCO₃ | 1.2 | 16 | 160 | 23 |
| 3 | NaHCO₃ | 2.2 | 16 | 160 | 35 |
| 4 | NaHCO₃ | 2.2 | 32 | 160 | 36 |
| 5 | Na₂CO₃ | 2.2 | 32 | 160 | 16 |
| 6 | K₂CO₃ | 2.2 | 32 | 160 | 17 |
| 7 | Ag₂CO₃ | 2.2 | 16 | 160 | Very few |
| 8 | Cs₂CO₃ | 2.2 | 40 | 160 | 36 |
| 9 | Azabicyclo | 2.2 | 16 | 160 | Non-reacted |
| | (DBU) | | | | |
| 10 | NaOAc | 2.2 | 40 | 160 | 17 |

By using the production method of the lamellarin derivative and the most suitable reaction condition provided by the above embodiment, and by using the appropriately substituted 3-nitrocoumarin as a starting material, it is demonstrated that the production method of the present application can quickly introduce methoxy of coumarin into the lamellarin derivative, and other substituents like chlorine and naphthyl can be introduced into the structure of the lamellarin by coupling reaction, thus, different lamellarin derivatives can be produced, such as the compounds represented by formulas (8a)-(8h) listed in the following Table 2, in addition, it is known from calculation that the yields of the lamellarin derivatives represented by formulas (8a)-(8h) are between 19 and 41 wt. %, and an average yield thereof is 20 wt. %.

Further, lamellarin derivatives represented by formulas (8a)-(8h) are demethylated by an overdose of boron tribromide (18 equivalents) to obtain hydroxyl-substituted lamellarin derivatives, represented by formulas (9a)-(9h), respectively, and the yields thereof is excellent.

TABLE 2
Lamellarin derivatives and yields thereof

| | | | Yield |
|---|---|---|---|
| Structure of lamellarin derivatives | R | Number | (%) |
| 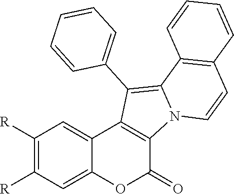 | R is OMe R is OH | Formula (8a) Formula (9a) | 30 91 |
| 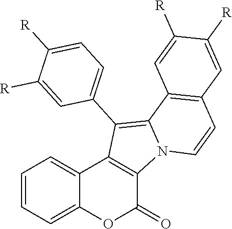 | R is OMe R is OH | Formula (8b) Formula (9b) | 35 87 |
| 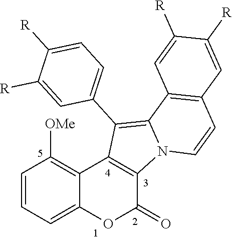 | R is OMe R is OH | Formula (8c) Formula (9c) | 22 92 |
| 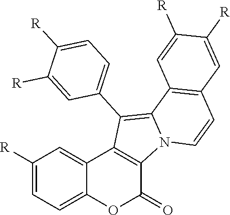 | R is OMe R is OH | Formula (8d) Formula (9d) | 27 89 |
| 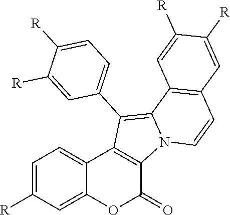 | R is OMe R is OH | Formula (8e) Formula (9e) | 37 90 |
| 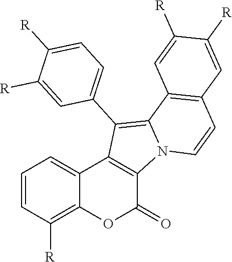 | R is OMe R is OH | Formula (8f) Formula (9f) | 19 83 |
| 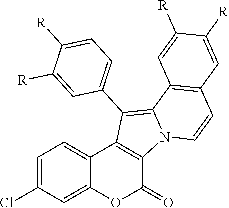 | R is OMe R is OH | Formula (8g) Formula (9g) | 41 90 |
| 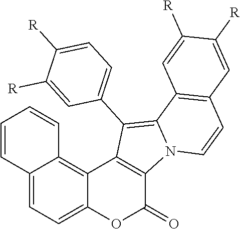 | R is OMe R is OH | Formula (8h) Formula (9h) | 23 84 |

In the production method for lamellarin H disclosed by a third embodiment of the present application, a main step is adopting the coupling reaction to yield a natural lamellarin as an alkaloid.

Please refer to the following reaction scheme (III):

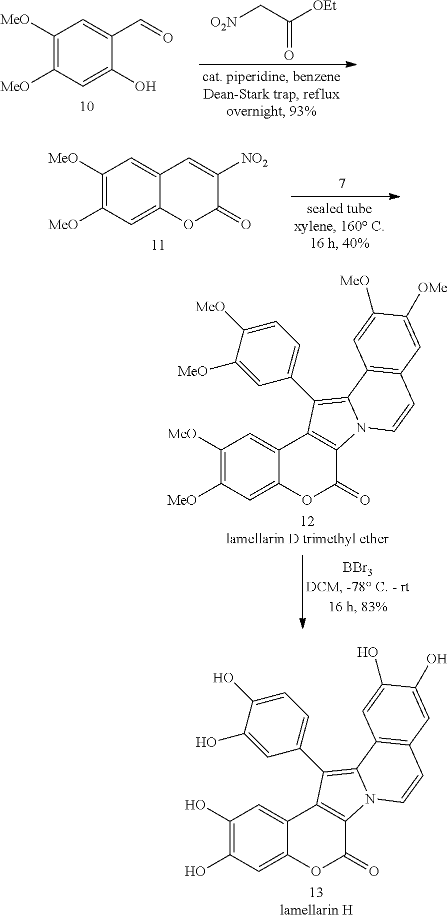 [SMILES: COc1cc(O)c(C=O)cc1OC ; CCOC(=O)C[N+](=O)[O-] ; COc1cc2cc([N+](=O)[O-])c(=O)oc2cc1OC ; COc1ccc(-c2c3c4cc(OC)c(OC)cc4oc(=O)c3n3ccc4cc(OC)c(OC)cc4c23)cc1OC ; O=c1oc2cc(O)c(O)cc2c2c(-c3ccc(O)c(O)c3)c3c4cc(O)c(O)cc4ccn3c12]

The commercially available compound represented by formula (10), i.e., o-hydroxy-2,4-dimethoxybenzaldehyde, and ethyl nitroacetate are performed with piperidine catalyzed condensation reaction in a Dean-Stark separator to acquire a compound represented by formula (11), i. e., 6,7-dimethoxy-3-nitrocoumarin, a yield of which is 93 wt. %, in which, the reaction condition includes addition of piperidine and benzene, and the reaction time is overnight. The compound represented by formula (11) and the compound represented by formula (7) are placed in a sealed tube for coupling reaction, in which, the reaction condition includes: addition of xylene, a reaction temperature of 160° C., and a reaction time of 16 hrs, to acquire lamellarin D trimethyl ether represented by formula (12), a yield of which is 40 wt. %, and a recovery rate of the compound represented by formula (7) is 26 wt. %. The compound represented by formula (12) is demethylated under a low temperature of approximately −78° C. by an overdose of a boron tribromide/dichloromethane solution with a reaction time of approximately 16 hrs, so that lamellarin H represented by formula (13) is acquired, and a yield thereof is 83 wt. %.

It is known from the third embodiment disclosed by the present application that it only requires three steps to synthesize lamellarin H by using the preparation method disclosed by the present application, and a total yield of the product is 31 wt. %. Compared with the prior art, the production method of the lamellarin disclosed by the present application obviously shortens the production path and greatly improves the production efficiency.

A fourth embodiment of the present application provides a production method of benzyloxy (OBn)-protected lamellarin D, and the method comprises the following steps:

(a) two compounds, i. e., 3-nitrocoumarin and 1-methylisoquinoline are firstly prepared;

(b) 3-nitrocoumarin and 1-methylisoquinoline are sealed in an alkaline environment for reaction to acquire a pentacyclic core of the lamellarin;

(c) bromination of the pentacyclic core of the lamellarin is carried out to acquire a brominated pentacyclic core; and

(d) Suzuki coupling reaction is conducted on the brominated pentacyclic core to acquire the OBn-protected lamellarin D.

Particularly, please refer to reaction schemes (IV)-(VI), which are reaction schemes required by the production method of the OBn-protected lamellarin D.

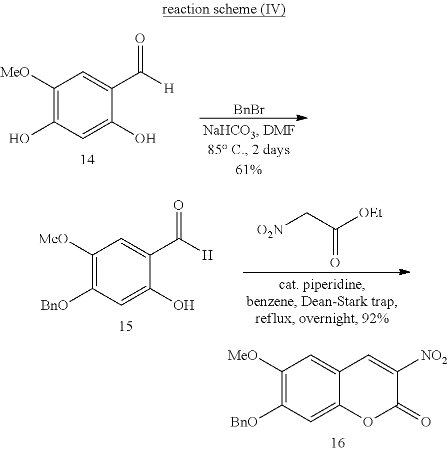

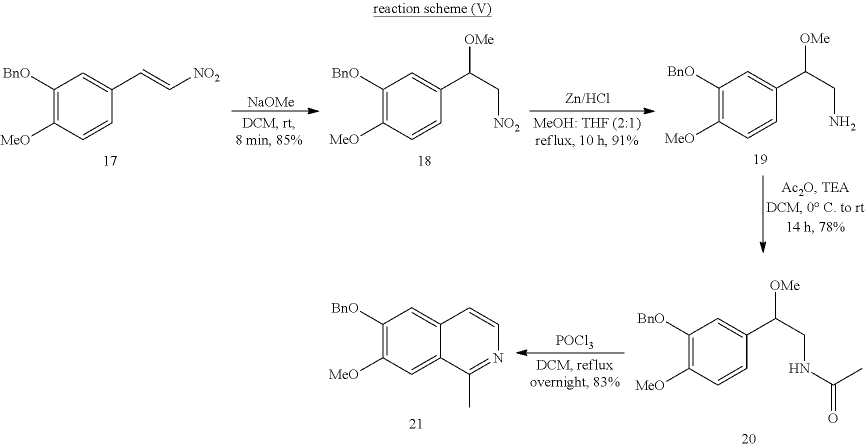

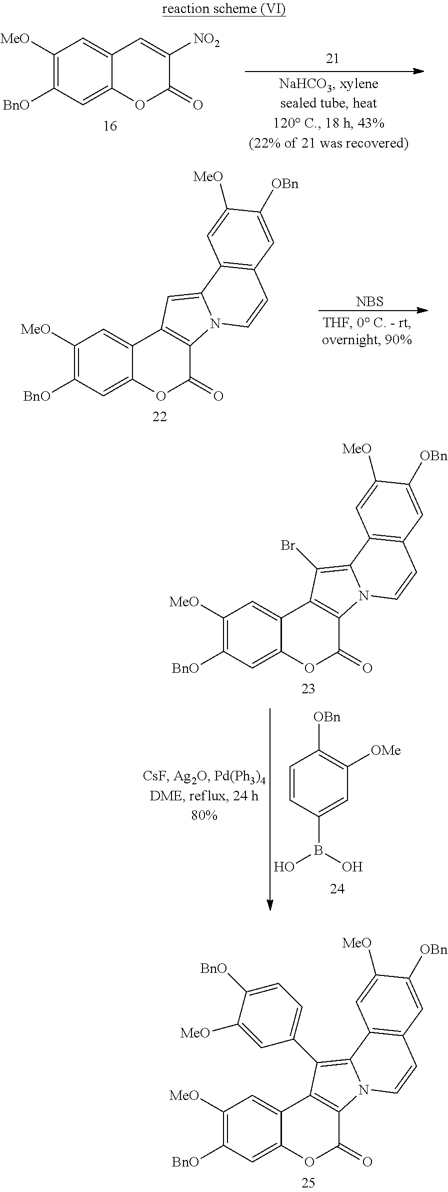

in which:

it is known from the reaction scheme (IV) that the compound represented by formula (16), i. e., 3-nitrocoumarin, is acquired by the following two reaction steps. First step: hydroxyl at C-4 position of an aldehyde compound represented by formula (14) is benzylated to acquire a compound represented by formula (15) with a yield of 61 wt. %, in which, the reaction condition includes: addition of sodium bicarbonate and dimethylformamide, a reaction temperature of 85° C., and a reaction time of 2 days; and second step: in a Dean-Stark separator, piperidine catalyzed condensation reaction of the compound represented by formula (15), i. e., a benzylated aldehyde, is carried out overnight in the presence ethyl nitroacetate to yield the compound represented by formula (16) with a yield of 92 wt. %, in which, the reaction condition includes addition of piperidine and benzene. A total yield of the compound represented by the formula (16) produced according to the reaction scheme (IV) is approximately 56 wt. %.

Please refer to reaction scheme (V), which includes the following reaction:

A compound represented by formula (21), i. e., 1-methylisoquinoline is synthesized by the following reaction steps: a n-position of a commercially available compound represented by formula (17), i. e., β-nitrostyrene, is conducted with methoxylation by sodium methylate at room temperature to acquire a compound represented by formula (18) with a yield of 85 wt. %, in which, the reaction condition includes addition of dichloromethane, and a reaction time is 8 mins.

The compound represented by formula (18) is reduced by a Zinc/hydrochloric acid solution to yield a compound represented by formula (19) with a yield of 91 wt. %, in which, the reaction condition includes addition of methanol/tetrahydrofuran solution (a volume ratio of MeOH to THF is 2:1) and a reaction time of 10 hrs.

The compound represented by formula (19) is treated by acetic anhydride to produce a corresponding compound represented by formula (20), a yield of which is 78 wt. %, in which, the reaction condition includes: addition of triethylamine and dichloromethane, a reaction temperature of between 0° C. and the room temperature, and a reaction time of 14 hrs.

Thereafter, Bischler-Napieralski cyclization of the compound represented by formula (20) is performed under the action of phosphorus oxychloride overnight to acquire compound represented by formula (21), a yield of which is 83 wt. %, in which, a reaction condition includes addition of dichloromethane.

Please refer to reaction scheme (VI) which explains reactions in steps (b)-(d) of the fourth embodiment of the present application, in which:

first, the compound represented by formula (16) reacts with the compound represented by formula (21) in a sealed circulating environment introduced with sodium bicarbonate to yield the pentacyclic core of the lamellarin represented by formula (22), a yield of which is approximately 43 wt. %, and a recovery rate of the compound represented by formula (21) is 22 wt. %, in which, the reaction condition includes addition of xylene, heating to 120° C., and a reaction time of 18 hrs.

Bromination of the compound represented by formula (22) is performed at room temperature by a N-bromosuccinimide (NBS)/tetrahydrofuran (THF) solution overnight to acquire a compound represented by formula (23), a yield of which is 90 wt. %.

The compound represented by formula (23) reacts with a compound represented by formula (24),i.e., phenylboronic acid for Suzuki coupling reflux reaction to synthesize the OBn-protected lamellarin D represented by formula (25), a yield of which is 80%, in which, the reaction condition includes addition of cesium fluoride, silver oxide, palladium tetraphosphite, and 1,2-dimethoxyethane (DME), and a reaction time is 24 hrs.

A fifth embodiment of the present application provides a method for producing lamellarin D, which comprises reactions illustrated in reaction scheme (VII):

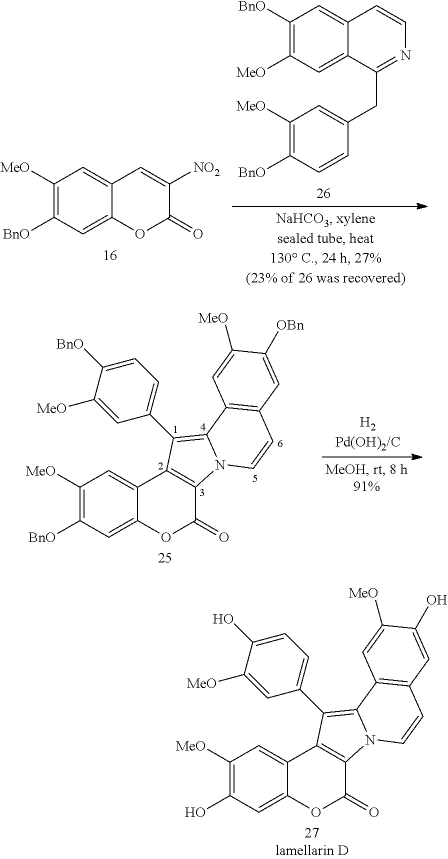 [SMILES: COc1cc2cc([N+](=O)[O-])c(=O)oc2cc1OCc1ccccc1 ; COc1cc(Cc2nccc3cc(OCc4ccccc4)c(OC)cc23)ccc1OCc1ccccc1 ; COc1cc(-c2c3c4cc(OC)c(OCc5ccccc5)cc4oc(=O)c3n3ccc4cc(OCc5ccccc5)c(OC)cc4c23)ccc1OCc1ccccc1 ; COc1cc(-c2c3c4cc(OC)c(O)cc4oc(=O)c3n3ccc4cc(O)c(OC)cc4c23)ccc1O]

in which:

The compound represented by formula (16) is coupled with a compound represented by formula (26) in a compound sealed tube, so that cyclization of two molecules produces the compound represented by formula (25), a yield of which is 27 wt. %, and a recovery rate of the compound represented by formula (26) is 23 wt. %, in which, a reaction condition includes: addition of sodium bicarbonate and xylene, heating to 130° C., and a reaction time is 24 hrs.

Under a hydrogen gas atmosphere, the compound represented by formula (25) is catalyzed by palladium hydroxide on carbon in ethanol for hydrogenolysis so as to acquire lamellarin D represented by formula (27), a yield of which is 91 wt. %, in which, a reaction condition includes: a reaction temperature of room temperature, a reaction time of 8 hrs, and addition of methanol.

Please refer to reaction scheme (VIII), a sixth embodiment of the present application provides another production method of a lamellarin to synthesize lamellarin 501 represented by formula (28), the reaction process of which is approximately the same as that of the fifth embodiment except that double bonds at positions of C-5 and C-6 (the numeral references 5 and 6 in the reaction scheme (VIII)) of the compound represented by formula (25) is catalyzed by palladium on carbon for hydrogenation, so that a compound represented by formula (28) is synthesized with a yield of 89 wt. %, in which, the reaction condition includes: hydrogen gas atmosphere, addition of methanol/ethyl acetate solution (a volume ratio of MeOH to EtOAc is 2:1), room temperature for reaction, and a reaction time is 18 hrs.

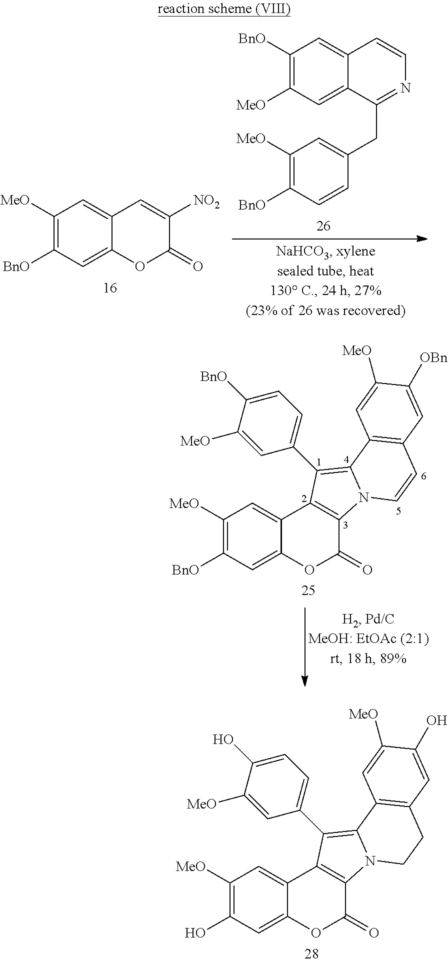 [SMILES: COc1cc2cc([N+](=O)[O-])c(=O)oc2cc1OCc1ccccc1 ; COc1cc(Cc2nccc3cc(OCc4ccccc4)c(OC)cc23)ccc1OCc1ccccc1 ; COc1cc(-c2c3c4cc(OC)c(OCc5ccccc5)cc4oc(=O)c3n3ccc4cc(OCc5ccccc5)c(OC)cc4c23)ccc1OCc1ccccc1 ; COc1cc(-c2c3n(c4c(=O)oc5cc(O)c(OC)cc5c24)CCc2cc(O)c(OC)cc2-3)ccc1O]

It is known from the explanation by the above embodiments that the production method of the lamellarin or the derivative thereof can synthesize the lamellarins and the derivatives thereof fluently, and significantly improve yields of the lamellarins and the derivatives thereof. As for the commercially available compound represented by formula (17), it only requires to 6 or 8 steps to complete the preparation of the lamellarin D, and a total yield of the lamellarin D is 12 wt. % and 14 wt. %, respectively, in addition, the method disclosed by the present application is able to greatly shorten the synthesis steps.

In order to further verify the present application, some examples are described hereinbelow for further explanation.

### Example 1 Synthesis of Core Structure of Lamellarin

209 mg (1.09 mmol, 1.2 equivalents) of 3-nitrocoumarin, 200 mg (0.912 mmol, 1 equivalent) of 1-benzylisoquinoline, and 243 mg (1.82 mmol, 2 equivalents) of aluminum chloride are collected and placed in a dry flask, 20 mL of toluene is added under a nitrogen gas atmosphere to form a mixture. The mixture is degassed and refluxed overnight, then cooled to the room temperature, a solvent is evaporated in vacuum to acquire a crude mixture. The crude mixture is purified using column chromatography to acquire 105 mg of a light yellow compound, i. e., core of lamellarin, a yield of which is 32 wt. %.

Characteristics of the light yellow compound are identified as follows:

Melting point (mp): 222-224° C.;

1H NMR (CDCl3, 400 MHz) δ 9.36 (d, J=7.2 Hz, 1H), 7.71 (d, J=8.0 Hz, 1H), 7.67-7.63 (m, 3H), 7.58-7.53 (m, 2H), 7.52-7.49 (m, 2H), 7.47-7.43 (m, 1H), 7.35 (td, J=7.6, 1.6 Hz, 1H), 7.25 (td, J=8.0, 1.2 Hz, 1H), 7.15 (d, J=7.2 Hz, 1H), 7.11 (dd, J=8.0, 1.6 Hz, 1H), 7.00 (td, J=8.4, 1.2 Hz, 1H);

13C NMR (CDCl3, 150 MHz) δ 155.3, 151.7, 135.6, 134.1, 130.9, 129.9, 129.7, 128.7, 128.7, 128.4, 128.2, 127.5, 127.3, 125.0, 124.4, 124.4, 124.2, 123.9, 117.9, 117.4, 114.3, 113.5, 109.4;

IR vmax (KBr) 3463, 2970, 1738, 1538, 1411, 1367, 1229, 1048, 968, 898 cm-1; and

HRMS (EI) m/z calculated value (calcd for) C₂₅H₁₅NO₂ [M+]: 361.1103, and experimental value (found): 361.1106.

It can be demonstrated from the above identification results that the light yellow compound is the core of the lamellarin.

### Example 2 Synthesis of Lamellarin Derivatives

3-nitrocoumarin derivative (1.2 equivalents), 1-benzylisoquinoline/papaverine, and sodium bicarbonate (2.2 equivalents) are mixed in 25 mL of a xylene solution then a mixture is placed into a dry and sealed tube which is then sealed by a Teflon sealing ring. The mixture is heated to 160° C. and maintained at such temperature for 16 hrs. After that, the mixture is cooled to room temperature, and a solvent is evaporated in vacuum so as to acquire a crude mixture. The crude mixture is then purified by using column chromatography to acquire a lamellarin derivative. During the purification, a recovery rate of papaverine as a starting material is approximately 20 wt. %.

Lamellarin derivatives acquired by this example have been further identified, results of which are listed in Table 3.

TABLE 3
Structures of lamellarin derivatives and characteristics thereof

| Structures of lamellarin derivatives | Characteristics |
|---|---|
| 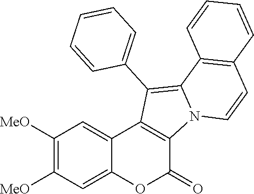 [SMILES: COc1cc2oc(=O)c3c(c(-c4ccccc4)c4c5ccccc5ccn43)c2cc1OC] | White solid; 155 mg; yield of 30%; melting point of 248-250° C.; 1H NMR (CDCl3, 400 MHz) δ 9.34 (d, J = 7.6 Hz, 1H), 7.74 (d, J = 8.0 Hz, 1H), 7.75-7.49 (m, 4H), 7.68 (s, 1H), 7.64 (s, 1H), 7.51 (td, J = 8.0, 1.2 Hz, 1H), 7.28 (td, J = 8.0, 1.2 Hz, 1H), 7.15 (d, J = 7.6 Hz, 1H), 6.97 (s, 1H), 6.53 (s, 1H), 3.92 (s, 3H), 3.42 (s, 3H); 13C NMR (CDCl3, 150 MHz) δ 155.6, 149.5, 146.6, 145.5, 135.8, 134.0, 131.4, 129.7, 129.4, 128.5, 128.1, 127.4, 127.3, 124.9, 124.5, 124.4, 113.1, 113.0, 109.7, 108.7, 104.8, 100.5, 56.1, 55.3; IR vmax (KBr) 3461, 2970, 1708, 1428, 1360, 1216, 1008, 788, 728 cm-1; HRMS (EI) m/z calcd for C27H19NO4 [M+] 421.1314, found 421.1310. |
| 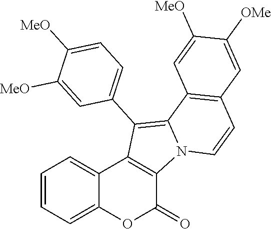 [SMILES: COc1ccc(-c2c3c4ccccc4oc(=O)c3n3ccc4cc(OC)c(OC)cc4c23)cc1OC] | Light white solid; 261 mg; yield of 35%; melting point of 250-252° C.; 1H NMR (CDCl3, 400 MHz) δ 9.28 (d, J = 7.2 Hz, 1H), 7.44 (dd, J = 8.0, 0.4 Hz, 1H), 7.37 (dd, J = 7.6, 1.6 Hz, 1H), 7.32 (td, J = 8.0, 2.8 Hz, 1H), 7.18 (dd, J = 6.4, 1.6 Hz, 1H), 7.14 (d, J = 8.4 Hz, 1H), 7.10- 7.09 (m, 3H), 7.68 (s, 1H), 7.04 (d, J = 8.0 Hz, 1H), 4.02 (s, 3H), 3.98 (s, 3H), 3.87 (s, 3H), 3.46 (s, 3H); 13C NMR (CDCl3, 150 MHz) δ 155.1, 151.7, 150.0, 149.9, 149.2, 149.1, 134.4, 128.6, 128.3, 128.0, 124.7, 124.1, 123.8, 123.7, 123.1, 119.1, 118.0, 117.2, 114.0, 112.7, 112.1, 112.0, 108.3, 107.3, 105.2, 56.1, 56.0, 55.9, 55.2; IR vmax (KBr) 3447, 3005, 1737, 1710, 1610, 1505, 1366, 1219, 1024, 753 cm-1; HRMS (EI) m/z calcd for C29H23NO6 [M+] 481.1525, found 481.1520. |
| 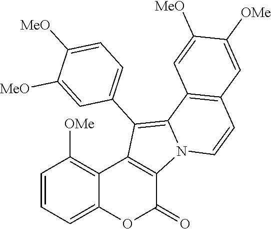 [SMILES: COc1ccc(-c2c3c4c(OC)cccc4oc(=O)c3n3ccc4cc(OC)c(OC)cc4c23)cc1OC] | Brown solid; 152 mg; yield of 22%; melting point of 234-236° C.; 1H 1H NMR (CDCl3, 400 MHz) δ 9.50 (d, J = 7.6 Hz, 1H), 7.34 (t, J = 8.0 Hz, 1H), 7.11-7.07 (m, 4H), 7.03 (s, 1H), 7.01 (d, J = 2.0 Hz, 1H), 6.74 (s, 1H), 6.62 (d, J = 8.0 Hz, 1H), 4.00 (s, 3H), 3.98 (s, 3H), 3.88 (s, 3H), 3.45 (s, 3H), 3.16 (s, 3H); 13C NMR (CDCl3, 150 MHz) δ 156.4, 155.3, 152.8, 149.8, 148.9, 148.8, 147.8, 135.2, 133.1, 128.6, 127.4, 124.9, 123.8, 123.1, 119.4, 114.9, 114.0, 113.1, 110.9, 109.9, 108.7, 108.6, 107.3, 105.9, 105.2, 56.2, 56.1, 55.9, 55.2, 54.7; IR vmax (KBr) 3602, 2969, 1714, 1608, 1435, 1365, 1226, 1027, 855, 787 cm-1; HRMS (EI) m/z calcd for C30H25NO7 [M+] 511.1631, found 511.1619. |
| 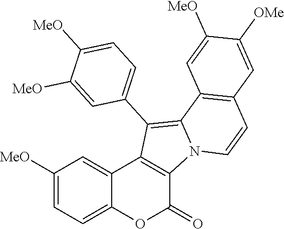 [SMILES: COc1ccc2oc(=O)c3c(c(-c4ccc(OC)c(OC)c4)c4c5cc(OC)c(OC)cc5ccn43)c2c1] | Light brown solid; 187 mg; yield of 27%; melting point of 252- 254° C.; 1H NMR (CDCl3, 400 MHz) δ 9.30 (d, J = 7.2 Hz, 1H), 7.37 (d, J = 8.8 Hz, 1H), 7.23 (dd, J = 8.4, 2.0 Hz, 1H), 7.19 (s, 1H), 7.16 (d, J = 8.0 Hz, 1H), 7.16 (d, J = 2.0 Hz, 1H), 7.15 (s, 1H), 7.11 (s, 1H), 7.09 (d, J = 7.6 Hz, 1H), 6.94 (dd, J = 8.8, 2.8 Hz, 1H), 6.83 (d, J = 2.8 Hz, 1H), 4.02 (s, 3H), 4.00 (s, 3H), 3.91 (s, 3H), 3.52 (s, 3H), 3.49 (s, 3H); 13C NMR (CDCl3, 100 MHz) δ 155.4, 155.3, 150.1, 149.9, 149.2, 149.1, 146.0, 134.2, 128.6, 128.0, 124.6, 123.9, 123.2, 119.1, 118.3, 118.1, 115.9, 114.2, 112.7, 112.0, 111.8, 108.6, 107.3, 106.7, 105.2, 56.2, 56.1, 55.9, 55.2, 55.1; IR vmax (KBr) 3461, 2933, 1713, 1690, 1479, 1411, 1225, 1045, 1005, 866, 799 cm-1; HRMS (EI) m/z calcd for C30H25NO7 [M+] 511.1631, found 511.1628. |
| 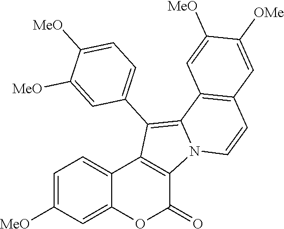 [SMILES: COc1ccc2c(c1)oc(=O)c1c2c(-c2ccc(OC)c(OC)c2)c2c3cc(OC)c(OC)cc3ccn21] | Yellow solid; 255 mg; yield of 37%; melting point of 256-258° C.; 1H NMR (CDCl3, 400 MHz) δ 9.26 (d, J = 7.6 Hz, 1H), 7.22 (d, J = 8.8 Hz, 1H), 7.19 (d, J = 1.6 Hz, 1H), 7.16 (d, J = 8.0 Hz, 1H), 7.11-7.10 (m, 3H), 7.07 (d, J = 7.6 Hz, 1H), 6.98-6.96 (m, 1H), 6.67 (d, J = 8.8, 2.4 Hz, 1H), 4.04 (s, 3H), 4.02 (s, 3H), 3.89 (s, 3H), 3.85 (s, 3H), 3.48 (s, 3H); 13C NMR (CDCl3, 150 MHz) δ 160.0, 155.3, 155.2, 150.1, 149.9, 149.2, 149.1, 134.6, 129.3, 128.1, 124.9, 124.8, 123.8, 123.3, 119.1, 114.1, 112.4, 112.0, 111.6, 111.3, 111.2, 107.5, 107.3, 105.3, 101.6, 56.1, 56.0, 55.9, 55.5, 55.2; IR vmax (KBr) 2936, 1707, 1617, 1431, 1314, 1225, 1139, 1046, 841, 756 cm-1; HRMS (EI) m/z calcd for C30H25NO7 [M+] 511.1631, found 511.1614. |
| 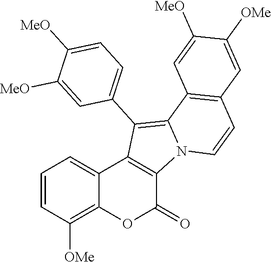 [SMILES: COc1ccc(-c2c3c4cccc(OC)c4oc(=O)c3n3ccc4cc(OC)c(OC)cc4c23)cc1OC] | Dark brown solid; 135 mg; yield of 19%; melting point of 254- 256° C.; 1H NMR (CDCl3, 400 MHz) δ 9.32 (d, J = 7.6 Hz, 1H), 7.20 (dd, J = 8.0, 1.6 Hz, 1H), 7.16 (d, J = 8.0 Hz, 1H), 7.13-7.10 (m, 1H), 7.11 (s, 1H), 7.10 (s, 1H), 7.09 (d, J = 7.2 Hz, 1H), 7.01 (d, J = 7.6 Hz, 1H), 6.69-6.91 (m, 2H), 4.04 (s, 3H), 4.00 (s, 3H), 3.99 (s, 3H), 3.90 (s, 3H), 3.48 (s, 3H); 13C NMR (CDCl3, 150 MHz) δ 154.6, 150.0, 149.9, 149.1, 149.0, 147.8, 141.2, 134.4, 128.7, 128.1, 124.7, 123.8, 123.6, 123.3, 119.2, 118.8, 115.9, 114.2, 112.7, 112.2, 112.0, 110.4, 108.5, 107.4, 105.3, 56.2, 56.1, 56.0, 55.9, 55.2; IR vmax (KBr) 3631, 2837, 1702, 1611, 1503, 1462, 1399, 1264, 1171, 1094 cm-1; HRMS (EI) m/z calcd for C30H25NO7 [M+] 511.1631, found 511.1630. |
| 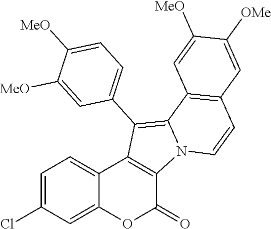 [SMILES: COc1ccc(-c2c3c4ccc(Cl)cc4oc(=O)c3n3ccc4cc(OC)c(OC)cc4c23)cc1OC] | White solid; 298 mg; yield of 41%; melting point of 296-298° C.; 1H NMR (CDCl3, 400 MHz) δ 9.24 (d, J = 7.6 Hz, 1H), 7.44 (d, J = 2.0 Hz, 1H), 7.22 (d, J = 8.4, 1H), 7.16 (s, 1H), 7.15 (d, J = 2.0 Hz, 1H), 7.11-7.10 (m, 4H), 7.03 (dd, J = 8.8, 2.4 Hz, 1H), 4.02 (s, 3H), 3.97 (s, 3H), 3.87 (s, 3H), 3.46 (s, 3H); 13C NMR (CDCl3, 150 MHz) δ 154.6, 152.0, 150.3, 150.0, 149.3, 149.2, 134.7, 133.8, 128.0, 127.6, 125.0, 124.8, 124.3, 123.7, 123.1, 119.1, 117.6, 116.7, 114.1, 113.1 112.1, 112.0, 108.0, 107.4, 105.2, 56.13, 56.10, 56.0, 55.3; IR vmax (KBr) 3452, 2970, 1711, 1504, 1428, 1385, 1210, 1030, 858, 755 cm-1; HRMS (El) m/z calcd for C29H22ClNO6 [M+] 515.1136, found 515.1132. |
| 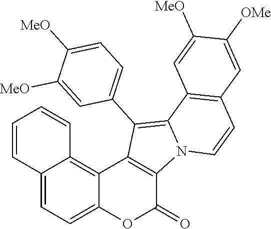 [SMILES: COc1ccc(-c2c3c4c(ccc5ccccc54)oc(=O)c3n3ccc4cc(OC)c(OC)cc4c23)cc1OC] | Brown solid; 149 mg; yield of 23%; melting point of 218-220° C.; 1H NMR (CDCl3, 400 MHz) δ 9.54 (d, J = 7.6 Hz, 1H), 7.88 (d, J = 8.8 Hz, 1H), 7.84 (d, J = 8.8 Hz, 1H), 7.80 (d, J = 8.0 Hz, 1H), 7.61 (d, J = 8.8 Hz, 1H), 7.35 (s, 1H), 7.31 (t, J = 7.2 Hz, 1H), 7.43 (d, J = 7.2 Hz, 1H), 7.14 (s, 1H), 7.13 (d, J = 6.4 Hz, 1H), 7.05 (d, J = 8.4 Hz, 1H), 7.01 (d, J = 1.6 Hz, 1H), 6.90 (td, J = 8.0, 1.6 Hz, 1H), 4.02 (s, 3H), 4.00 (s, 3H), 3.70 (s, 3H), 3.53 (s, 3H); 13C NMR (CDCl3, 150 MHz) δ 155.3, 150.6, 150.0, 149.7, 149.0, 134.6, 130.9, 130.5, 130.0, 129.3, 128.4, 127.7, 127.9, 125.1, 125.0, 124.7, 124.6, 124.5, 123.1, 119.2, 117.8, 115.7, 113.4, 113.2, 113.0, 111.8, 110.3, 107.5, 105.9, 56.3, 56.1, 55.9, 55.4; IR vmax (KBr) 3439, 3004, 1715, 1617, 1416, 1365, 1226, 1049, 989, 810, 752 cm-1; HRMS (EI) m/z calcd for C33H25NO6 [M+] 531.1682, found 531.1679. |

### REFERENCE

Kluza, J.; Gallego, M.-A.; Loyens, A.; Beauvillain, J.-C.; Sousa-Faro, J.-M. F.; Cuevas, C.; Marchetti, P.; Bailly, C. Cancer Res. 2006, 66, 3177-3187.
Ballot, C.; Kluza, J.; Lancel, S.; Martoriati, A.; Hassoun, S. M.; Mortier, L.; Vienne, J.-C.; Briand, G.; Formstecher, P.; Bailly, C.; Neviere, R.; Marchetti, P. Apoptosis 2010, 15, 769-781.
Facompre', M.; Tardy, C.; Bal-Mahieu, C.; Colson, P.; Perez, C.; Manzanares, I.; Cuevas, C.; Bailly, C. Cancer Res. 2003, 63, 7392-7399.
Marco, E.; Laine, W.; Tardy, C.; Lansiaux, A.; Iwao, M.; Ishibashi, F.; Bailly, C.; Gago, F. J. Med. Chem. 2005, 48, 3796-3807.
Khiati, S.; Seol, Y; Agama, K.; Rosa, I. D.; Agrawal, S.; Fesen, K.; Zhang, H.; Neuman, K. C.; Pommier, Y. Mol. Pharmacol. 2014, 86, 193-199.
Ploypradith, P.; Mahidol, M.; Sahakitpichan, P.; Wongbundit, S.; Ruchirawat, S. Angew. Chem. Int. Ed. 2004, 43, 866-866.

The invention claimed is:

1. A method for producing a compound presented by formula (I), 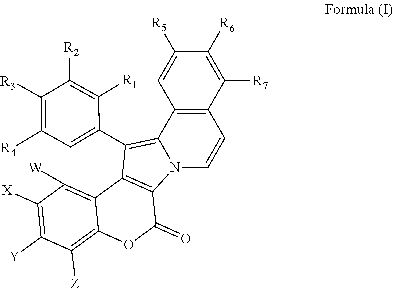
the method comprising performing intermolecular cyclization reaction between a 3-nitrocoumarin derivative and a papaverine derivative under a pre-determined reaction condition to acquire a lamellarin or a derivative thereof, wherein
the reaction condition comprises use of at least one solvent and heating, and the solvent is selected from the group consisting of an aromatic hydrocarbon, an ether, and a halogenated hydrocarbon;
W is selected from the group consisting of hydrogen, chlorine, bromine, fluorine, methoxy, methyl, and cyano;
X, Y, and Z are respectively and independently selected from the group consisting of hydrogen, benzyloxy, hydroxyl, chlorine, bromine, fluorine, trifluoromethyl, methoxy, methyl, and cyano;
R1, R2, R7 are hydrogen respectively;
R3, R5, R6 are respectively and independently selected from the group consisting of methoxy, benzyloxy, hydroxyl, and hydrogen; and
R4 is selected from the group consisting of methoxy, hydroxyl, and hydrogen.

2. The method of claim 1, wherein
a structure of the 3-nitrocoumarin derivative is represented by formula (II): 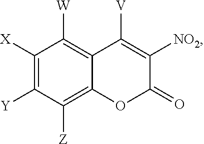
in which, V is selected from the group consisting of hydrogen and chlorine; and
a structure of the papaverine derivative is represented by formula (III):

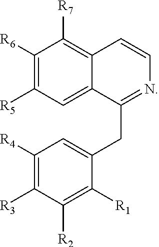

3. The method of claim 2, wherein the 3-nitrocoumarin derivative is selected from the group consisting of 3-nitrocoumarin, 6-methoxy-7-benzyloxy-3-nitrocoumarin, and 6,7-dimethoxy-3-nitrocoumarin.

4. The method of claim 2, wherein the papaverine derivative is selected from the group consisting of 1-(3,4-dimethoxybenzyl)-6,7-dimethoxyisoquinoline, and 1-(3-benzyloxy-4-methoxy)-6-methoxy-7-benzyloxyisoquinoline.

5. The method of claim 1, wherein the intermolecular cyclization reaction is performed within a sealed space.

6. The method of claim 1, wherein a reaction temperature is at least 120° C.

7. The method of claim 6, wherein the reaction temperature is between 120 and 160° C.

8. The method of claim 1, wherein the reaction condition further comprises addition of an alkaline substance.

9. The method of claim 8, wherein the alkaline substance is selected from the group consisting of sodium bicarbonate, cesium carbonate, sodium carbonate, and potassium carbonate.

10. The method of claim 1, wherein the solvent is selected from the group consisting of xylene, toluene, tetrahydrofuran, and 1,2-dichloroethane.

11. The method of claim 1, comprising the following steps:
step a: performing intermolecular cyclization reaction between 6-methoxy-7-benzyloxy-3-nitrocoumarin and 1-(3-benzyloxy-4-methoxy)-6-methoxy-7-benzyloxyisoquinoline in a sealed alkaline environment to acquire a compound having a pentacyclic core;
step b: performing catalytic hydrogenolysis on the compound having the pentacyclic core under a hydrogen gas atmosphere to acquire the compound represented by formula (I);
wherein
a catalyst is selected from the group consisting of palladium hydroxide on carbon and palladium on carbon;
X, R2, and R3 represent methoxy respectively;
Y, R1, and R4 represent hydroxyl respectively; and
R5 represents hydrogen.

12. The method of claim 1, wherein 6,7-dimethoxy-3-nitrocoumarin and 1-(3,4-dimethoxybenzyl)-6,7-dimethoxyisoquinoline are performed with the intermolecular cyclization reaction in a sealed environment to acquire the compound represented by formula (I), in which, X, Y, R1, R2, R3, and R4 represent methoxy respectively, and R5 represents hydrogen.

13. The method of claim 1, comprising the following steps:
step a: performing intermolecular cyclization reaction between 6,7-dimethoxy-3-nitrocoumarin and 1-(3,4-dimethoxybenzyl)-6,7-dimethoxyisoquinoline in a sealed environment to acquire a compound having a pentacyclic core; and
step b: demethylating the compound having the pentacyclic core acquired from step a to remove methoxy therefrom so as to acquire the compound represented by formula (I);
wherein
X, Y, R1, R2, R3, and R4 represent hydroxyl respectively; and
R5 represents hydrogen.

14. The method of claim 13, wherein in step b, a boron tribromide/dichloromethane solution is added for demethylation.

15. The method of claim 2, wherein intermolecular cyclization reaction is performed between the compound represented by formula (II) and the compound represented by formula (III) in a sealed environment to acquire the compound represented by formula (I), wherein
W is hydrogen;
X is selected from the group consisting of methoxy, hydrogen, and hydroxyl;
Y is selected from the group consisting of methoxy, hydroxyl, hydrogen, and chlorine;
Z is selected from the group consisting of methoxy, hydroxyl, and hydrogen;
R1, R2, and R7 are hydrogen respectively; and
R3, R4, R5, and R6 are respectively and independently selected from the group consisting of methoxy, hydroxyl, and hydrogen.

16. The method of claim 15, wherein demethylation is performed after the intermolecular cyclization reaction is completed to acquire the compound represented by formula (I), wherein
W is hydrogen;
X is selected from the group consisting of hydrogen and hydroxyl;
Y is selected from the group consisting of hydroxyl, hydrogen, and chlorine;
Z is selected from the group consisting of hydroxyl and hydrogen;
R1, R2, and R7 are hydrogen respectively; and
R3, R4, R5, and R6 are independently and respectively selected from the group consisting of hydroxyl and hydrogen.

17. The method of claim 16, wherein the reaction condition further comprises addition of an alkaline substance.

18. The method of claim 17, wherein the alkaline substance is selected from the group consisting of sodium bicarbonate, cesium carbonate, sodium carbonate, and potassium carbonate.
